(19) United States
(12) Patent Application Publication
(10) Pub. No.: US 2015/0290447 A1
(43) Pub. Date: Oct. 15, 2015

(54) Medical Tubing Tether Device

(71) Applicant: Wendy Gorowicz, Thornton, CO (US)

(72) Inventor: Wendy Gorowicz, Thornton, CO (US)

(21) Appl. No.: 14/252,841

(22) Filed: Apr. 15, 2014

(51) Int. Cl. A61M 39/10

(52) U.S. Cl. CPC A61M 39/10

(57) ABSTRACT

A medical tubing tether device secures medical tubing to a patient to prevent the medical tubing from being inadvertently removed from the patient's nose. The device includes a tether comprising a loop configured to be positioned around a person's neck. A fastener is attached to said tether. The fastener is configured for removably coupling medical tubing to the tether.

### BACKGROUND OF THE DISCLOSURE

### Field of the Disclosure

The disclosure relates to medical tubing supports and more particularly pertains to a new medical tubing support for securing medical tubing to a patient to prevent the medical tubing from being inadvertently removed from the patient's nose.

### SUMMARY OF THE DISCLOSURE

An embodiment of the disclosure meets the needs presented above by generally comprising a tether comprising a loop configured to be positioned around a person's neck. A fastener is attached to said tether. The fastener is configured for removably coupling medical tubing to the tether.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a side perspective view of a medical tubing tether device according to an embodiment of the disclosure.

FIG. 2 is a top view of an embodiment of the disclosure.

FIG. 3 is a detailed view of an adhesive of an embodiment of the disclosure attaching medical tubing to the tether.

FIG. 4 is a front view of an embodiment of the disclosure in use.

FIG. 5 is a top view of an alternative embodiment of the disclosure.

### DESCRIPTION OF THE PREFERRED EMBODIMENT

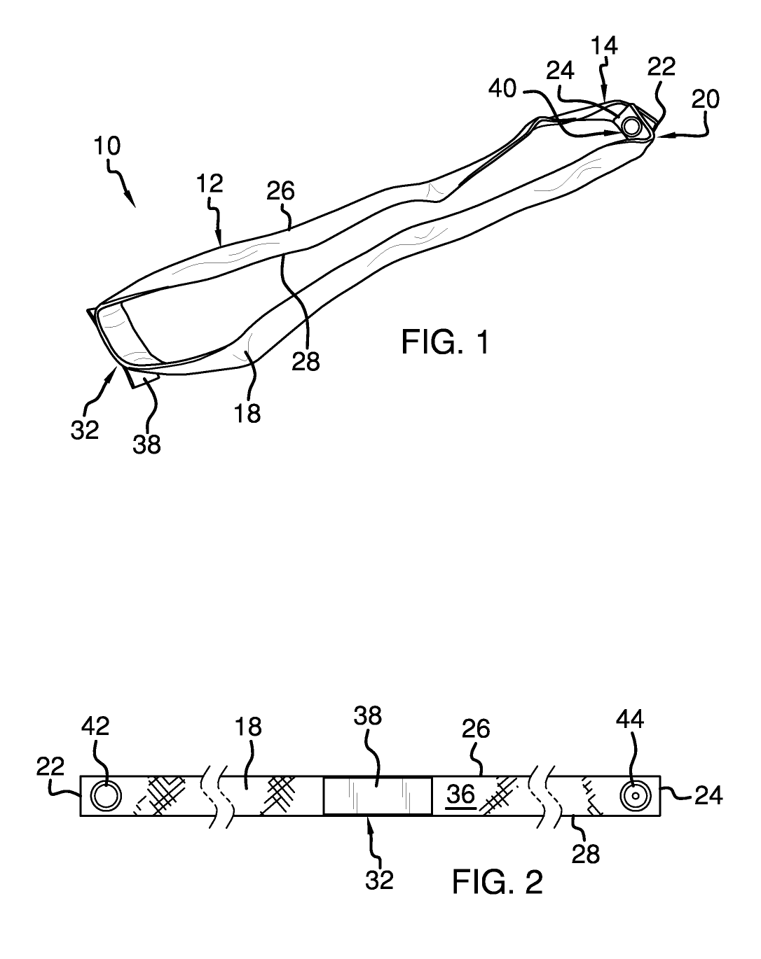

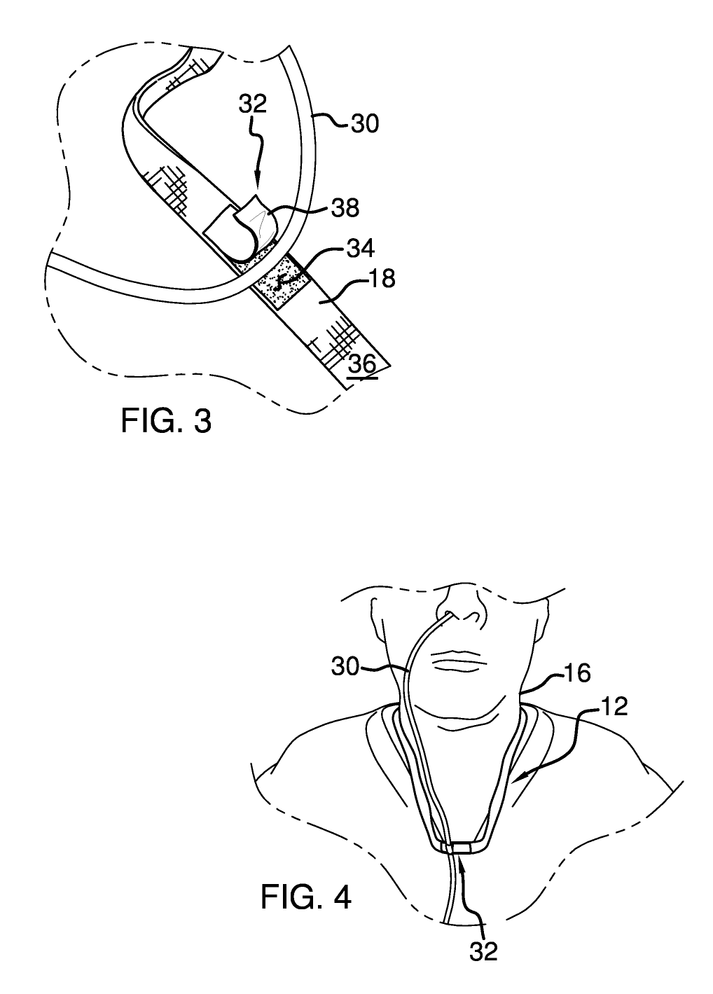

With reference now to the drawings, and in particular to FIGS. 1 through 5 thereof, a new medical tubing support embodying the principles and concepts of an embodiment of the disclosure and generally designated by the reference numeral 10 will be described.

As best illustrated in FIGS. 1 through 5, the medical tubing tether device 10 generally comprises a tether 12. The tether 12 comprises a loop 14 configured to be positioned around a person's neck 16. The tether 12 may comprise a flexible strap 18 made of cloth, leather or like material. The tether 12 has a first break 20 therein to define a first free end 22 and a second free end 24. The tether 12 includes a first lateral side 26 and a second lateral side 28 each coupled to and extending between the first 22 and second 24 free ends. The tether 12 may have a width extending from the first lateral side 26 to the second lateral side 28 measuring between approximately 1.0 cm and 2.5 cm.

At least one medical tube 30 is provided. The medical tube 30 may include a feeding tube, an oxygen tube or the like. A fastener 32 is attached to the tether 12. The fastener 32 removably couples each medical tube 30 to the tether 12. The fastener 12 may be centrally positioned between the first free end 22 and the second free end 24, as shown in FIGS. 1 and 2. The fastener 32 may include an adhesive 34 positioned on an outer surface 36 of the tether 12. The fastener 32 may further include a cover 38 removably attached to the adhesive 34. The tether 12 is attachable to each medical tube 30 such that each medical tube 30 is positionable between the cover 38 and the adhesive 34 to secure each medical tube 30 to the tether 12.

A coupler 40 is attached to the tether 12. The coupler 40 releasably couples the first 22 and second 24 free ends together. The coupler 40 includes a first mating member 42 positioned proximate the first free end 22 and a second mating member 44 positioned proximate the second free end 24. The first mating member 42 is engageable with the second mating member 44 to releasably attach the first free end 22 to the second free end 24. The coupler 40 may comprise snaps.

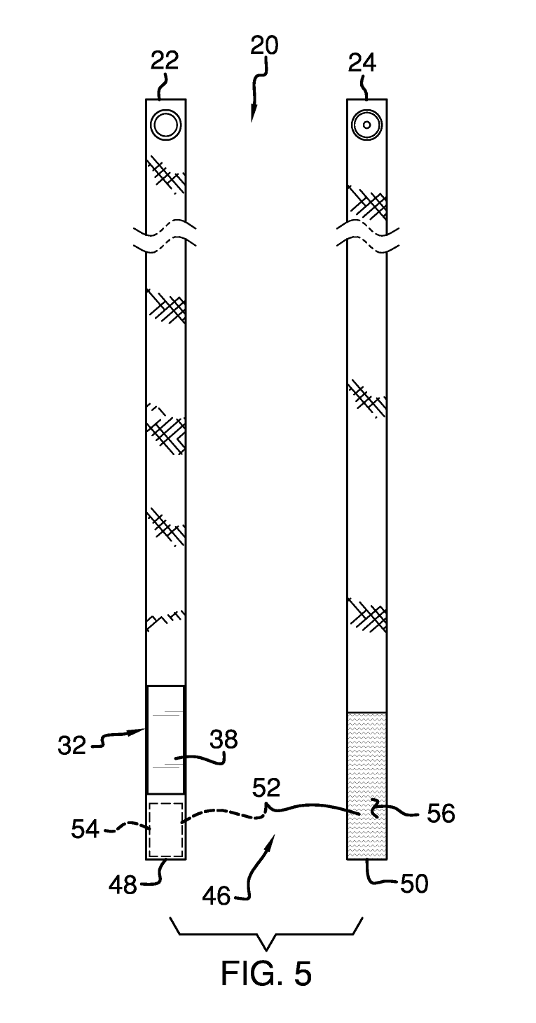

An alternative embodiment is shown in FIG. 5. Unless mutually exclusive or otherwise described, the alternative embodiment is identical to the embodiments shown in FIGS. 1-4. As shown in FIG. 5, the tether 12 may have a second break 46 therein to define a third free end 48 and a fourth free end 50. A connector 52 is attached to the tether 12. The connector 52 releasably couples the third 48 and fourth 50 free ends together. The connector 52 may comprise complementary portions 54, 56 of hook and loop fastener. One of the complementary portions 54, 56 of hook and loop fastener may be positioned adjacent to the fastener 32. Each of the complementary portions 54, 56 of hook and loop fastener may comprise elongated strips.

In use, the tether 12 is secured around the neck 16 of a patient using coupler 40. The medical tube 30 is positioned on the adhesive 34 and is secured to the tether 12 by positioning the cover 38 over the medical tube 30 and onto the adhesive 34. When sufficient force engages the medical tube 30, either the first 22 and second 24 free ends and/or the third 48 and fourth 50 free ends will release from each other. In this manner, the device 10 helps to prevent inadvertent removal of the medical tube 30 from the patient's nasal cavity and redistributes force exerted on the medical tube 30 onto the tether 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. A medical tubing tether device comprising:
a tether comprising a loop configured to be positioned around a person's neck; and
a fastener attached to said tether, said fastener being configured for removably coupling medical tubing to said tether.

2. The device of claim 1, further comprising:
said tether having a first break therein to define a first free end and a second free end; and
a coupler attached to said tether, said coupler releasably coupling said first and second free ends together.

3. The device of claim 1, further comprising wherein said coupler includes a first mating member positioned proximate said first free end and a second mating member positioned proximate said second free end, said first mating member being engageable with said second mating member to releasably attach said first free end to said second free end.

4. The device of claim 1, further comprising wherein said fastener includes an adhesive positioned on an outer surface of said tether.

5. The device of claim 4, further comprising wherein said fastener further includes a cover removably attached to said adhesive, said tether being configured for attachment to the medical tubing such that the medical tubing is positionable between said cover and said adhesive to secure the medical tubing to said tether.

6. The device of claim 2, further comprising said fastener being centrally positioned between said first free end and said second free end.

7. The device of claim 3, further comprising wherein said coupler comprises snaps.

8. The device of claim 1, further comprising wherein said tether comprises a strap.

9. The device of claim 2, further comprising:
said tether having a second break therein to define a third free end and a fourth free end; and
a connector attached to said tether, said connector releasably coupling said third and fourth free ends together.

10. The device of claim 9, further comprising wherein said connector comprises complementary portions of hook and loop fastener.

11. The device of claim 10, further comprising wherein one of said complementary portions of hook and loop fastener is positioned adjacent to said fastener.

12. A medical tubing tether device comprising:
a tether being comprised of a flexible material, said tether comprising a loop configured to be positioned around a person's neck, said tether comprising a strap, said tether having a first break therein to define a first free end and a second free end, said tether having a second break therein to define a third free end and a fourth free end;
a coupler attached to said tether, said coupler releasably coupling said first and second free ends together, said coupler including a first mating member positioned proximate said first free end and a second mating member positioned proximate said second free end, said first mating member being engageable with said second mating member to releasably attach said first free end to said second free end, said coupler comprising snaps;
a fastener attached to said tether, said fastener being configured for removably coupling medical tubing to said tether, said fastener including an adhesive positioned on an outer surface of said tether, said fastener further including a cover removably attached to said adhesive, said tether being configured for attachment to the medical tubing such that the medical tubing is positionable between said cover and said adhesive to secure the medical tubing to said tether; and
a connector attached to said tether, said connector releasably coupling said third and fourth free ends together, said connector comprising complementary portions of hook and loop fastener, one of said complementary portions of hook and loop fastener being positioned adjacent to said fastener, each of said complementary portions of hook and loop fastener being elongated strips.

13. A medical tubing tether system comprising:
a tether comprising a loop configured to be positioned around a person's neck;
at least one medical tube; and
a fastener attached to said tether, said fastener removably coupling each said medical tube to said tether.

14. The system of claim 13, further comprising:
said tether comprising a strap, said tether having a first break therein to define a first free end and a second free end, said tether having a second break therein to define a third free end and a fourth free end;
a coupler attached to said tether, said coupler releasably coupling said first and second free ends together, said coupler including a first mating member positioned proximate said first free end and a second mating member positioned proximate said second free end, said first mating member being engageable with said second mating member to releasably attach said first free end to said second free end, said coupler comprising snaps;
said fastener including an adhesive positioned on an outer surface of said tether, said fastener further including a cover removably attached to said adhesive, said tether being attachable to each said medical tube such that each said medical tube is positionable between said cover and said adhesive to secure each said medical tube to said tether; and
a connector attached to said tether, said connector releasably coupling said third and fourth free ends together, said connector comprising complementary portions of hook and loop fastener, one of said complementary portions of hook and loop fastener being positioned adjacent to said fastener, each of said complementary portions of hook and loop fastener being elongated strips.
